(19) United States
(12) Patent Application Publication
(10) Pub. No.: US 2024/0285342 A1
(43) Pub. Date: Aug. 29, 2024

(54) SUPPRESSING RELAXATION OSCILLATIONS FOR LITHOTRIPSY LASERS

(71) Applicant: Boston Scientific Scimed Inc., Maple Grove, MN (US)

(72) Inventors: Jian Zhang, Lancaster, MA (US); Steven Yihlih Peng, Fremont, CA (US); Howard D. Simms, JR., Shoreview, MN (US); Noah Lyden, Shoreview, MN (US)

(73) Assignee: Boston Scientific Scimed Inc., Maple Grove, MN (US)

(21) Appl. No.: 18/590,617

(22) Filed: Feb. 28, 2024

Related U.S. Application Data
(60) Provisional application No. 63/448,869, filed on Feb. 28, 2023.

(51) Int. Cl. A61B 18/26; A61B 17/00; A61B 18/00

(52) U.S. Cl. CPC A61B 18/26; A61B 2017/00172; A61B 2017/0019; A61B 2018/00732

(57) ABSTRACT

Described are methods, systems, and techniques for a laser system controller, such as a controller for a laser used in a medical device. The controller can be configured to generate a pulse-width modulated control signal, the pulse-width modulated control signal comprising a plurality of electrical pulses separated by a temporal delay; and communicating the pulse width modulated control signal to an optical pump, wherein the optical pump, responsive to the pulse width modulated control signal, generates optical pump light and wherein a lasing medium, when exposed to the optical pump light, generates a laser beam.

### CROSS-REFERENCE TO RELATED APPLICATIONS

This application claims the benefit of U.S. Provisional Patent Application Ser. No. 63/448,869 filed Mar. 7, 2023, the disclosure of which is incorporated herein by reference.

### TECHNICAL FIELD

Aspects of the present disclosure generally relate to laser systems and methods of using laser systems for medical applications.

### BACKGROUND

In medical applications, laser energy is used in many procedures. Non-limiting examples of such procedures include incision, excision, resection, vaporization, ablation, fragmentation, coagulation, hemostasis, denaturation, etc., of various body tissues. In some medical procedures, lasers having, for example, a wavelength of about 2100 nanometers (nm) (or 2.1 micrometers (μm)) may be used since the energy of this wavelength is highly absorbed by water, a constituent of virtually all tissues. In ureteroscopic laser lithotripsy (URS), laser energy is used to disintegrate stones in the urinary tract of a subject (patient, etc.). In some applications, laser lithotripsy may be performed using a number of different types of lasers, such as, for example, a Holmium YAG (Ho:YAG) laser, a Thulium YAG (Tm:YAG) laser, or a Chromium (Cr) Thulium (Tm) Holmium (Ho) YAG (CTH:YAG) laser. Such lasers provide a relatively high fragmentation efficiency for different types of stones.

Although the CTH:YAG laser has been the benchmark laser system for URS since its introduction more than twenty years ago, optical coating damage of the cavity mirrors is still one of the dominant laser failure modes. The damaged optical coating can significantly reduce the laser output, and the constant energy control system's feedback loop can speed up the degradation till the system is out of normal operational range.

The systems and methods of the current disclosure may rectify some of these or other deficiencies in known laser systems. The scope of the current disclosure, however, is defined by the attached claims, and not by the ability to solve any specific problem.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

The disclosure can be implemented as a method for a laser system controller. The method can comprise generating a pulse-width modulated control signal, the pulse-width modulated control signal comprising a plurality of electrical pulses separated by a temporal delay; and communicating the pulse width modulated control signal to an optical pump, wherein the optical pump, responsive to the pulse width modulated control signal, generates optical pump light and wherein a lasing medium, when exposed to the optical pump light, generates a laser beam.

In further embodiments of the method, the temporal delay between a first electrical pulse and a second electrical pulse of the plurality of electrical pulses is less than the temporal delay between the second electrical pulse and a third electrical pulse of the plurality of electrical pulses.

In further embodiments of the method, a magnitude of a first electrical pulse is less than a magnitude of a subsequent electrical pulse of the plurality of electrical pulses.

In further embodiments of the method, the temporal delay between a first electrical pulse and a second electrical pulse of the plurality of electrical pulses is less than the temporal delay between the second electrical pulse and a third electrical pulse of the plurality of electrical pulses, and wherein a magnitude of the first electrical pulse is less than a magnitude of the second electrical pulse of the plurality of electrical pulses.

In further embodiments of the method, a frequency of the pulse width modulated control signal is between 4 and 25 kilohertz.

In further embodiments of the method, the pulse width modulated control signal comprises a plurality of sets of electrical pulses, each set of electrical pulses of the plurality of sets of electrical pulses comprising a plurality of temporally spaced apart electrical pulses.

The disclosure can be implemented as a laser system. The laser system can comprise a controller configured to send a pulse width modulated control signal to one or more optical pumps to cause the optical pumps to generate optical pump light; a lasing medium arranged to output a laser beam in response to the optical pump light; and a pump chamber configured to direct the optical pump light to the lasing medium, wherein the pulse width modulated control signal comprises a plurality of temporally spaced apart electrical pulse components configured to dampen a relaxation oscillation of the laser beam.

In further embodiments of the laser system, the temporal delay between a first electrical pulse and a second electrical pulse of the plurality of electrical pulses is less than the temporal delay between the second electrical pulse and a third electrical pulse of the plurality of electrical pulses.

In further embodiments of the laser system, a magnitude of a first electrical pulse is less than a magnitude of a subsequent electrical pulse of the plurality of electrical pulses.

In further embodiments of the laser system, the temporal delay between a first electrical pulse and a second electrical pulse of the plurality of electrical pulses is less than the temporal delay between the second electrical pulse and a third electrical pulse of the plurality of electrical pulses, and wherein a magnitude of the first electrical pulse is less than a magnitude of the second electrical pulse of the plurality of electrical pulses.

In further embodiments of the laser system, the frequency of the pulse width modulated control signal is between 4 and 25 kilohertz.

In further embodiments of the laser system, the pulse width modulated control signal comprises a plurality of sets of electrical pulses, each set of electrical pulses of the plurality of sets of electrical pulses comprising a plurality of temporally spaced apart electrical pulses.

In further embodiments, the laser system can comprise a saturable absorber disposed of in an optical path of the laser beam.

In further embodiments of the laser system, the lasing medium includes one of Ho:YAG, Tm:YAG, Tm:Ho:YAG, Er:YAG, Er:YLF, Nd:YAG, Thulium fiber laser, and CTH:YAG.

The disclosure can be implemented as a non-transitory computer-readable storage medium. The computer-readable storage medium can include instructions that, when executed by a computer or a controller for a laser system, cause the computer or the controller to generate a pulse-width modulated control signal, the pulse-width modulated control signal comprising a plurality of electrical pulses separated by a temporal delay; and communicate the pulse width modulated control signal to an optical pump, wherein the optical pump, responsive to the pulse width modulated control signal, generates optical pump light and wherein a lasing medium, when exposed to the optical pump light, generates a laser beam.

In further embodiments of the computer-readable storage medium, the temporal delay between a first electrical pulse and a second electrical pulse of the plurality of electrical pulses is less than the temporal delay between the second electrical pulse and a third electrical pulse of the plurality of electrical pulses.

In further embodiments of the computer-readable storage medium, a magnitude of a first electrical pulse is less than that of a subsequent electrical pulse of the plurality of electrical pulses.

In further embodiments of the computer-readable storage medium, the temporal delay between a first electrical pulse and a second electrical pulse of the plurality of electrical pulses is less than the temporal delay between the second electrical pulse and a third electrical pulse of the plurality of electrical pulses, and wherein a magnitude of the first electrical pulse is less than a magnitude of the second electrical pulse of the plurality of electrical pulses.

In further embodiments of the computer-readable storage medium, the frequency of the pulse width modulated control signal is between 4 and 25 kilohertz.

In further embodiments of the computer-readable storage medium, the pulse width modulated control signal comprises a plurality of sets of electrical pulses, each set of electrical pulses of the plurality of sets of electrical pulses comprising a plurality of temporally spaced apart electrical pulses.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 illustrates an aspect of the subject matter in accordance with one embodiment.

FIG. 2A illustrates an aspect of the subject matter in accordance with one embodiment.

FIG. 2B illustrates an aspect of the subject matter in accordance with one embodiment.

FIG. 3 illustrates an aspect of the subject matter in accordance with one embodiment.

FIG. 4 illustrates an aspect of the subject matter in accordance with one embodiment.

FIG. 5 illustrates an aspect of the subject matter in accordance with one embodiment.

FIG. 6A illustrates an aspect of the subject matter in accordance with one embodiment.

FIG. 6B illustrates an aspect of the subject matter in accordance with one embodiment.

FIG. 7 illustrates an aspect of the subject matter in accordance with one embodiment.

FIG. 8 illustrates a routine 800 in accordance with one embodiment.

FIG. 9 illustrates a computer-readable storage medium 900 in accordance with one embodiment.

FIG. 10 illustrates an aspect of the subject matter in accordance with one embodiment.

FIG. 11A illustrates an aspect of the subject matter in accordance with one embodiment.

FIG. 11B illustrates an aspect of the subject matter in accordance with one embodiment.

FIG. 11C illustrates an aspect of the subject matter in accordance with one embodiment.

FIG. 11D illustrates an aspect of the subject matter in accordance with one embodiment.

FIG. 11E illustrates an aspect of the subject matter in accordance with one embodiment.

FIG. 12A illustrates an aspect of the subject matter in accordance with one embodiment.

FIG. 12B illustrates an aspect of the subject matter in accordance with one embodiment.

FIG. 12C illustrates an aspect of the subject matter in accordance with one embodiment.

FIG. 12D illustrates an aspect of the subject matter in accordance with one embodiment.

FIG. 12E illustrates an aspect of the subject matter in accordance with one embodiment.

FIG. 12F illustrates an aspect of the subject matter in accordance with one embodiment.

FIG. 13A illustrates an aspect of the subject matter in accordance with one embodiment.

FIG. 13B illustrates an aspect of the subject matter in accordance with one embodiment.

FIG. 13C illustrates an aspect of the subject matter in accordance with one embodiment.

### DETAILED DESCRIPTION

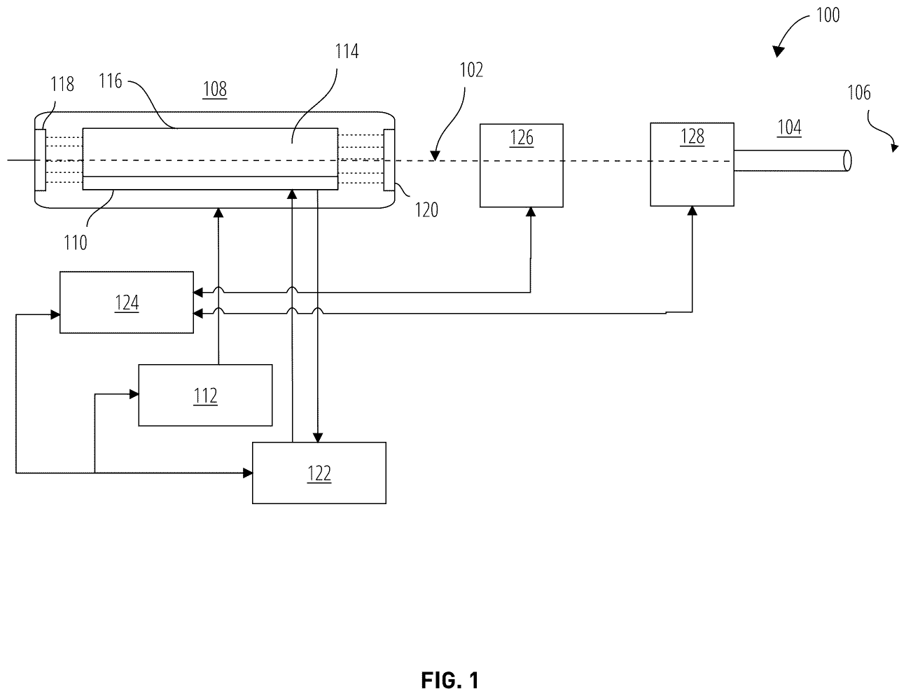

FIG. 1 is a schematic illustration of an exemplary laser system 100 of the present disclosure. Laser system 100 is suitable for generating and delivering pulses of laser radiation (laser beam 102) directly or through a conventional laser delivery system (e.g., optical fiber 104) to a target site 106 within a subject's body (e.g., a stone within the subject's urinary tract, or the like). Laser system 100 includes an oscillator 108 configured to generate and deliver a laser pulse in the form of laser beam 102 to the target site 106 through the optical fiber 104. The oscillator 108 includes a solid-state lasing medium 114 that includes one or more lasing ions (e.g., Ho, Cr, Th, Er, etc.) doped in various concentrations within a host material (e.g., YAG, etc.). In some embodiments, the lasing medium 114 may have a relatively long emission and fluorescence lifetime. As is known to a person of skill in the art, fluorescence lifetime is a measure of time the electrons of the lasing medium 114 spends in an excited state before returning to its ground state by emitting a photon.

The oscillator 108 may include an optical pump 110 for optically exciting the lasing medium 114 to produce laser beam 102. The optical pump 110 may include any known type of device (e.g., flashlamp, arc lamp, electrically pumped LED, laser diode, diode-pumped laser, solid state crystal or fiber laser, etc.) used to excite the lasing medium 114 optically. The optical pump 110 may be powered by a power supply 112 that includes components to operate the optical pump 110. These components may include, among others, a capacitor for energy storage and discharge, an inductor for pulse shaping, and a trigger circuit for ionizing the optical pump 110.

The power supply 112 may be configured to deliver electrical pulses from the capacitor to operate the optical pump 110. A pump chamber 116 may house both the lasing medium 114 and the optical pump 110 and allow optical radiation to transfer from the optical pump 110 to the lasing medium 114. The pump chamber 116 may also allow for the efficient cooling of both the lasing medium 114 and the optical pump 110. The laser oscillator 108 may include a housing, upon which is mounted the pump chamber 116, a fully (or substantially fully) reflective optic 118, and a partially reflective optic 120. Both the fully reflective optic 118 and the partially reflective optic 120 may include one or more optical components (lens, mirrors, etc.) of various physical shapes and may be coated for reflection and/or transmittance of various wavelength(s) of radiation. In some embodiments, these reflective optics 118 and 120 may be positioned in adjustable mounts that allow these components to be centered on the path of a laser beam traversing the solid-state lasing medium 114.

A cooling system 122 may supply a coolant to cool heat-producing components of the laser system 100. The liquid or gaseous coolant may be recirculated through the pump chamber 116 in a closed loop to cool the heat-producing components of the oscillator 108. In some embodiments, the cooling system 122 may be cooled by air. In some embodiments, other components of the laser system 100 (e.g., power supply 112) may also be cooled by the coolant of the cooling system 122. It is also contemplated that, in some embodiments, the cooling system 122 may be used to heat the components of the oscillator 108.

The operation of the laser system 100 may be controlled by a controller 124. As known in the art, the controller 124 may include a central processing unit (CPU) and other components that facilitate control of the laser system 100 (e.g., power supply 112, cooling system 122, oscillator 108, etc.) based on user input and/or feedback from sensors/detectors that monitor the performance of the laser system 100. For example, based on user input, controller 124 may control the power supply 112 to generate electrical pulses of desired characteristics (amplitude, frequency, pulse duration, etc.) to operate the optical pump 110. And based on the measured temperature of the oscillator 108, controller 124 may control the operation of the cooling system 122. Since the functions of a controller 124 of a laser system are well known in the art, they are not discussed extensively herein.

During the operation of the laser system 100, controller 124 controls the power supply 112 to direct a current/voltage waveform or an electrical pulse having desired characteristics (amplitude, duration, magnitude, etc.) to the optical pump 110. The optical energy generated by the optical pump 110, as a result, is coupled to the lasing medium 114. For optimal coupling, the optical pump 110 may be arranged around and positioned in close contact with the lasing medium 114. In some embodiments, coupling of optical energy into the lasing medium 114 may be improved through reflections off the interior surfaces of pump chamber 116. The optical energy from the optical pump 110 raises the energy level of the electrons in the lasing medium 114 to achieve population inversion. Population inversion occurs when more of the electrons of the lasing medium 114 exist in their excited state (i.e., higher energy state) than their ground state (i.e., normal, or low energy state). As population inversion is achieved, one or more wavelengths of optical radiation pass multiple times through the lasing medium 114 and are reflected off both the fully reflective optic 118 and the partially reflective optic 120. When the excitation level of the electrons reaches a threshold value (referred to in laser physics as the lasing threshold or laser threshold), laser pulses forming laser beam 102 are generated and emitted along the longitudinal axis of the oscillator 108.

A portion of the emitted laser pulses of laser beam 102 may be sampled by an optical detector 126 to facilitate monitoring and control of the laser system 100. For example, the controller 124 may use signals from the optical detector 126 (that are indicative of the emitted laser beam) to monitor and control the operation of the oscillator 108, the power supply 112, the cooling system 122, and other components of the laser system 100. The emitted laser beam 102 may then be directed through one or more optical coupling elements 128 to condition the subsequent outgoing pulses to enter the proximal end of the optical fiber 104. The laser beam 102 is then transmitted through the optical fiber 104 to its distal end that may be placed in contact with (or in close proximity to) a stone (not shown) at the target site 106. At target site 106, the impinging laser beam 102 may fragment the stone.

The laser system 100 of FIG. 1 may also include additional components (such as, for example, controllers, mirrors, focusing elements, beam blocking devices, Q-switching, or mode-locking elements, etc.) that are well known to people of ordinary skill in the art, and therefore, not illustrated in FIG. 1 and not discussed herein. For example, in some embodiments, the laser system 100 may be further controlled using reflected radiation (or radiation that is rescattered, reemitted, changed in wavelength by the stone material, etc.) from the stone, and transmitted back to the proximal end through the optical fiber 104. Further, in some embodiments, a visible aiming beam (e.g., low-power semiconductor diode laser, helium-neon (HeNe) laser, etc.) may be provided to assist in focusing the emitted laser beam 102 at the target site 106.

The characteristics (energy, pulse width, power, frequency, etc.) of the emitted laser beam 102 may depend on the characteristics of the optical pulses generated by the optical pump 110. The characteristics of these optical pulses may depend on the electrical pulses directed to the optical pump 110 from the power supply 112.

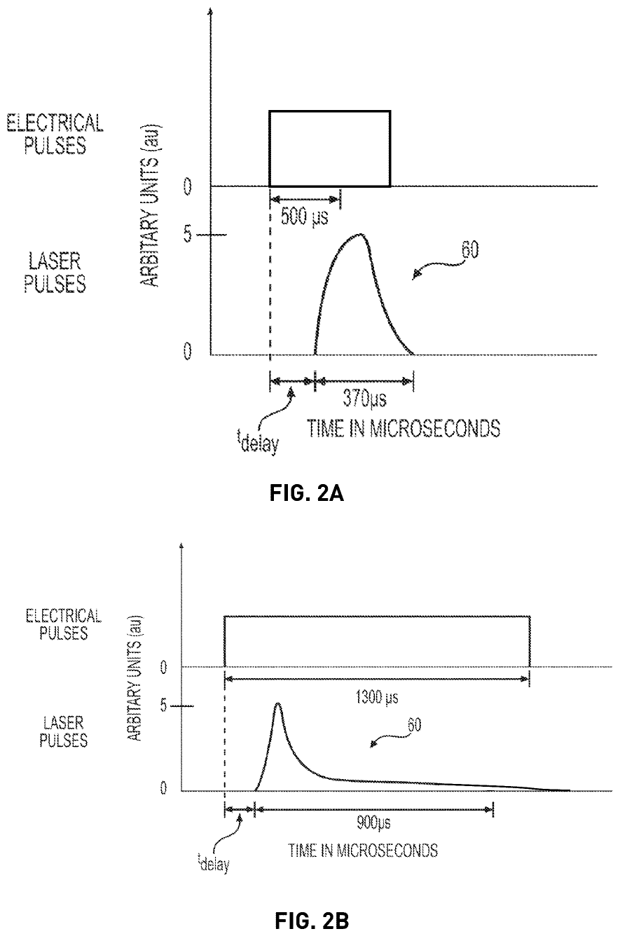

FIG. 2A and FIG. 2B are simplified charts that illustrate exemplary relationships between an electrical pulse to the optical pump 110 and the resulting pulses of laser beam 102 from the oscillator 108 in a standard operating mode. In these figures, the upper portion illustrates the electrical pulse directed to the optical pump 110, and the lower portion illustrates the resulting laser pulse itself. FIG. 2A illustrates the case when a relatively short electrical pulse is directed to the optical pump 110, while FIG. 2B illustrates the case when a relatively longer electrical pulse is directed to the optical pump 110.

Although the electrical pulses in both FIG. 2A and FIG. 2B are illustrated as rectangular pulses; this is merely a simplification. In reality, these pulses are not square but are rounded due to several factors (e.g., losses in the components, etc.). In contrast with such typical electrical pulses, the corresponding pulses of laser beam 102 have a characteristic shark-fin shape. That is, the laser pulses of laser beam 102 have a high initial peak followed by a steep decline. The area of the curve in FIG. 2A and FIG. 2B is indicative of the energy of the corresponding laser beam 102. As can be seen from both FIG. 2A and FIG. 2B, there is a finite time delay (t_{delay}) between the start of an electrical pulse activating optical pump 110 and the resulting pulse of laser beam 102. This time delay is related to the time it takes for the lasing medium 114 to reach the lasing threshold. Simplistically, the energy of the electrical pulse in the time period t_{delay} is used to raise the excitation level of the electrons in the lasing medium 114 to the lasing threshold. When this threshold is reached or exceeded, a laser beam 102 emanates. The time delay may be a function of the energy of the electrical pulse and the laser beam 102.

Further, as can be seen in both FIG. 2A and FIG. 2B, the emitted pulses of laser beam 102 are shorter (in time scale) than the electrical pulse that generates it. For example, in FIG. 2A, the electrical pulse has a duration of about 500 microseconds (μs) and the pulse of laser beam 102 that results from this electrical pulse is only about 370 μs, and in FIG. 2B, while the electrical pulse has a duration of about 1300 μs the resulting pulse of laser beam 102 only has a duration of about 900 μs. This difference in duration is a result of the energy losses that occur in the oscillator 108 as well as due to a reduction in fluorescence lifetime due to stronger pumping and thermal effects. Additionally, as can be seen by comparing FIG. 2A and FIG. 2B, because of the rapidly decaying shape of the laser pulse curve, the increase in energy (area under the laser pulse curve) resulting from a longer electrical pulse is not significant.

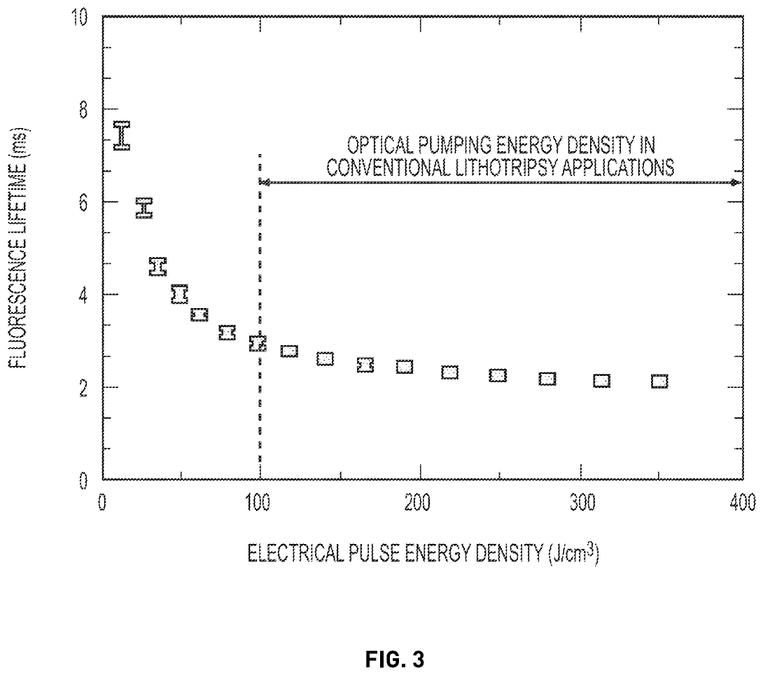

Conventionally, to increase the energy of the laser pulse, the power (or magnitude) of the electrical pulse (or pumping energy) is increased. Increasing the power of the electrical pulse (and thereby the optical pumping energy) increases the magnitude of the initial peak of the corresponding laser pulse. Further, it is well known that the fluorescence lifetime of a lasing medium may strongly depend on the optical pumping energy. FIG. 3 illustrates the fluorescence lifetime of a CTH:YAG lasing medium as a function of optical pumping energy density. As can be seen from FIG. 3, when a lasing medium 114 is pumped at low energy levels, the fluorescence lifetime of the lasing medium 114 is substantially greater than when it is pumped at high energy levels. For example, when the optical pumping energy density is increased from about 30 Jules (J) per centimeter (cm) cubed (J/cm³) to about 300 J/cm³, the fluorescence lifetime of the lasing medium decreases from about 6 milliseconds (ms) to about 2 ms. Conventional laser systems use optical pumping energy between about 100-300 J/cm³ for lithotripsy applications. Thus, the efficiency of the laser is reduced due to a shorter fluorescence lifetime, especially for long laser pulses with a pulse duration in the range of the fluorescence lifetime.

Additionally, the higher magnitude initial peak resulting from a high-power electrical pulse may cause the energy of the laser beam 102 to, at least momentarily, exceed a desired value. This momentary increase in laser energy may damage the optical components of the laser system 100 and cause undesirable effects such as retropulsion and large stone fragments (which may have to be removed using additional medical devices such as, e.g., retrieval baskets).

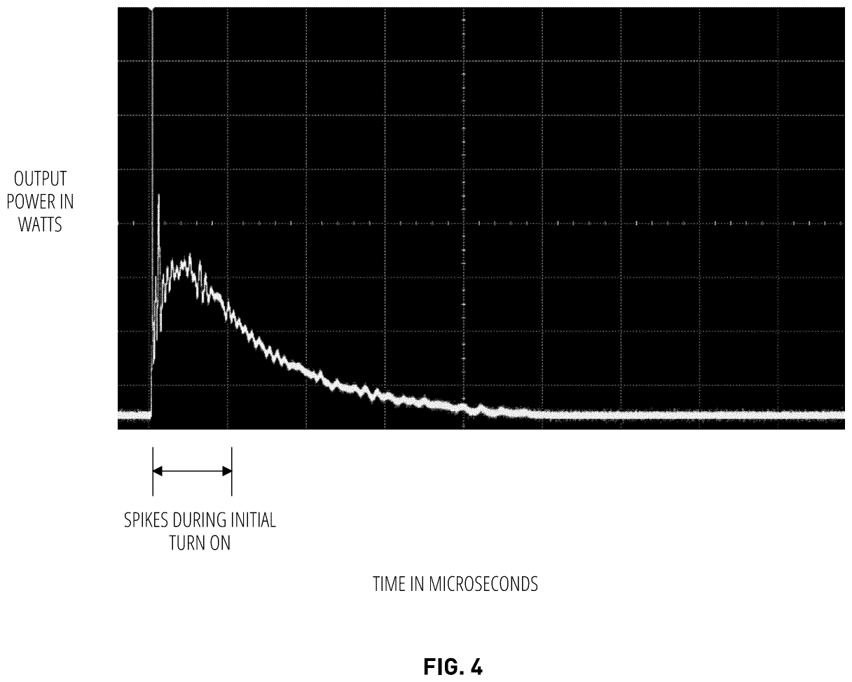

Additionally, relaxation-oscillation-related laser power spikes at the beginning of each laser pulse could have much higher peak power than the average peak power calculated as the laser pulse energy over the pulse width. FIG. 4 illustrates the output power for a laser beam 102. As can be seen, spikes in the output power of the laser beam 102 occur when power to the optical pump 110 is turned. After the emission of a few pulses, the output power “relaxes” to a steady state. This phenomenon is referred to as relaxation oscillations. Relaxation oscillations can be caused by the initial turn-on of the laser system 100 or even by changes in desired power. For example, the upper-state lifetime of CTH:YAG laser is approximately 8.5 ms, which is much longer than the laser resonator's damping time (e.g., approximately 6 μs). As a result, the laser dynamics are such that even changes in pump power can lead to relaxation oscillations.

These spikes in output power can exceed the laser-induced damage threshold (LIDT) of the cavity optics (e.g., pump chamber 116, reflective optic 118, partially reflective optic 120, etc.) and contribute to the coating damage.

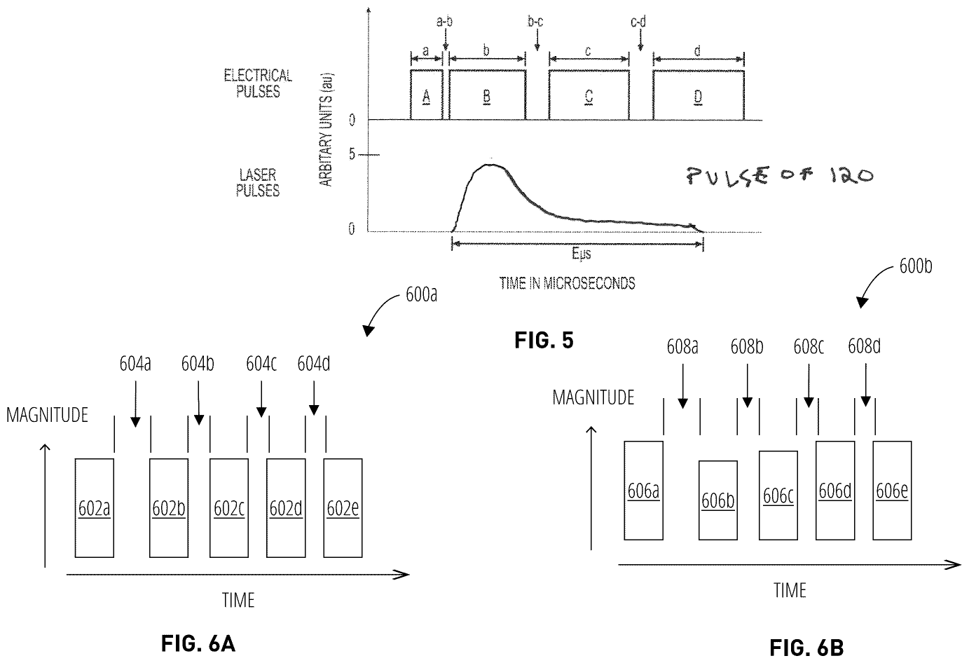

FIG. 5 is a simplified chart that illustrates the relationship between a pulse width modulated electrical pulses exciting optical pump 110 and the resulting pulses of laser beam 102, according to at least one embodiment of the present disclosure. In this embodiment, multiple temporally spaced-apart pulse width modulated (PWM) components of electrical pulses (marked A, B, C, D) are directed from the power supply 112 to the optical pump 110 such that a pulse of laser beam 102 results. As can be seen, this pulse exhibits reduced or damped relaxation oscillations. That is, the spikes during the initiation of laser beam 102 resulting from the electrical pulses A, B, C, and D are less than conventional electrical pulses used to excite optical pump 110. In some embodiments, the initial electrical pulse to the optical pump 110 may be a pre-pulse (marked A) that is configured to raise the energy level of the lasing medium 114 to a value that is close to, but just below, the lasing threshold (e.g., to about 80-99% of the lasing threshold). Pre-pulse A does not result in an emitted laser beam 102 since the excitation caused by pre-pulse A does not reach the lasing threshold. However, at the end of the pre-pulse A at time ti, the electrons of the lasing medium 114 may be primed to emit radiation in the form of pulses of light (e.g., laser beam 102) upon further addition of a small amount of energy. A sequence of additional electrical pulses (B, C, D) is then provided to the optical pump 110 in a short intervals to initiate the pulse of partially reflective optic 120 from the oscillator 108. In general, the plurality of pulses A, B, C, and D may be spaced apart such that a subsequent electrical pulse is within the fluorescence lifetime of its immediately preceding pulse (i.e., pulse B is within the fluorescence lifetime of pulse A, pulse C is within the fluorescence lifetime of pulse B, etc.).

In general, the shape of the pulse of laser beam 102 depends on the characteristics of the laser system 100 (e.g., power, spacing, duration, etc. of the electrical pulses, the material of the lasing medium, etc.). In some embodiments, the duty cycle of the PWM electrical pulses A, B, C, and D is dynamically manipulated to form a pulse of partially reflective optic 120 having reduced or damped relaxation oscillations, as depicted in FIG. 5.

In some embodiments, the electrical pulse of the set of spaced-apart electrical pulses may be formed using a pulse width modulation (PWM) formula such that the laser beam 102 resulting from the individual laser pulses generated by the electrical pulses has a smoother turn-on profile, or rather, exhibits damped relaxation oscillations. For example, pulses A, B, C, and D can be formed to have a PWM between 4 and 25 kilohertz (kHz). As a specific example, earlier pulses can have a larger delay between the pulses than later pulses such that the laser beam 102 exhibits reduced relaxation oscillations. As another example, earlier pulses (e.g., pulse B) can have lower power than later pulses such that the laser beam 102 exhibits reduced relaxation oscillations.

FIG. 6A illustrates a series of electrical pulses 600a, which can be generated by controller 124 and transmitted to optical pump 110, causing optical pump 110 to generate pump light to irradiate lasing medium 114 as described above. Electrical pulses 600a can include several electrical pulses. This embodiment depicts electrical pulses 602a, 602b, 602c, 602d, and 602e. As can be seen, the electrical pulses 602a through 602e are separated by a temporal delay (e.g., delay 604a, delay 604b, delay 604c, or delay 604d. For example, electrical pulse 602a and electrical pulse 602b are separated by delay 604a. Likewise, electrical pulse 602b and electrical pulse 602c are separated by delay 604b. In some embodiments, delays 604a through delay 604d are not the same. For example, the delay can decrease with time.

FIG. 6B illustrates a series of electrical pulses 600b, which can be generated by controller 124 and transmitted to optical pump 110, causing optical pump 110 to generate pump light to irradiate lasing medium 114 as described above. Electrical pulses 600b can include several electrical pulses. This embodiment depicts electrical pulses 606a, 606b, 606c, 606d, and 606e. As can be seen, the electrical pulses 606a through 606e are separated by a temporal delay (e.g., delay 608a, delay 608b, delay 608c, or delay 608d. For example, electrical pulse 606a and electrical pulse 606b are separated by delay 608a. Likewise, electrical pulse 606b and electrical pulse 606c are separated by delay 608b. In some embodiments, the magnitude of delays 604a through delay 604d are not the same. For example, the magnitude can increase with time.

With some embodiments, a pulse width modulated control signal can be generated comprising electrical pulses having characteristics like both electrical pulses 602a through 602e and electrical pulses 606a through 606e. That is, the electrical pulses can both have different temporal delays and magnitudes as depicted and described.

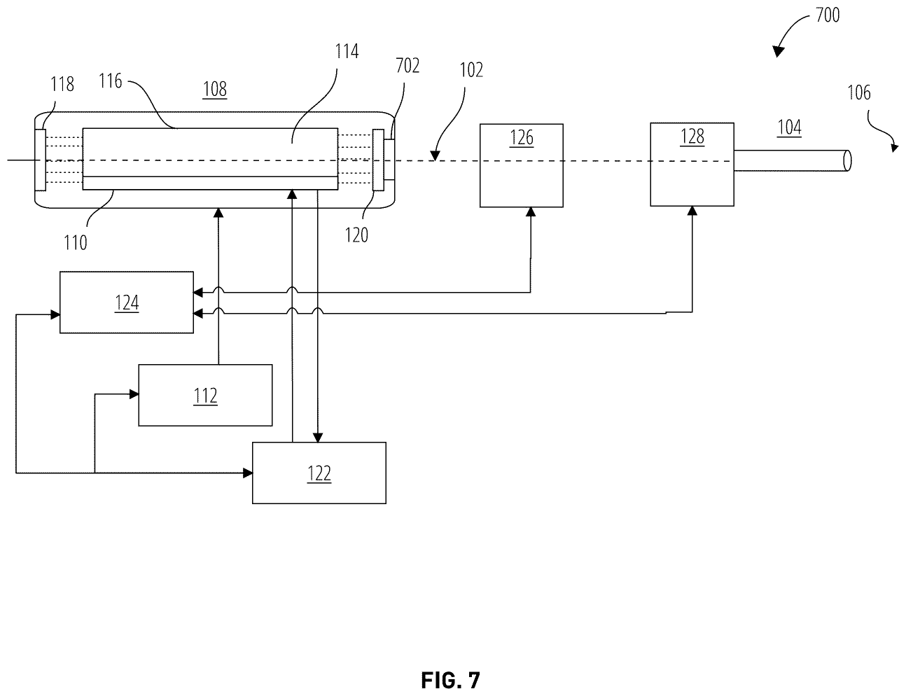

FIG. 7 is a schematic illustration of an exemplary laser system 700 of the present disclosure. Laser system 700 is like laser system 100 and is suitable for generating and delivering pulses of laser radiation directly or through a conventional laser delivery system to a target site 106 within a subject's body (e.g., a stone within the subject's urinary tract or the like). Laser system 700 includes the same components of the laser system 100 in addition to a saturable absorber 702 disposed within the oscillator 108 and arranged to dampen or reduce the relaxation oscillations of laser beam 102. It is to be appreciated that the saturable absorber 702 is configured to absorb light. However, the light absorbed decreases with the incident light intensity. As such, the saturable absorber 702 is configured to form, in combination with the other components of the laser system 700 and the above-described control method, a quasi-continuous waveform (e.g., laser beam 102).

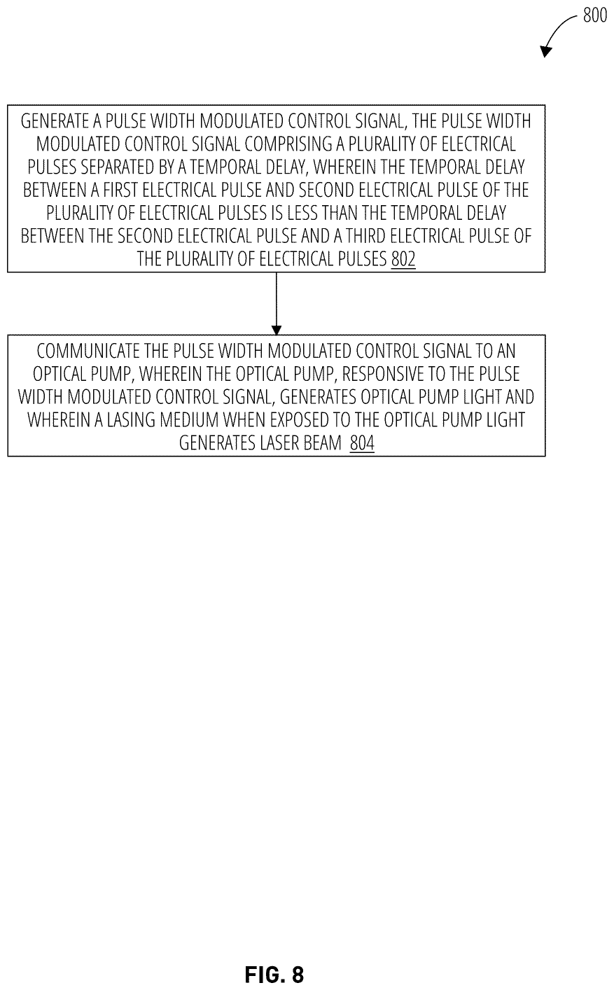

FIG. 8 depicts a routine 800, which can be implemented by a controller of a laser system, such as, for example, controller 124 of the laser system 100. Routine 800 can begin at block 802. At block 802, routine 800 generates a pulse width modulated control signal, the pulse width modulated control signal comprising a plurality of electrical pulses separated by a temporal delay, wherein the time delay between a first electrical pulse and a second electrical pulse of the plurality of electrical pulses is less than the temporal delay between the second electrical pulse and a third electrical pulse of the plurality of electrical pulses. For example, controller 124 can generate a pulse width modulated control signal including several electrical pulses (e.g., electrical pulses 600a, electrical pulses 600b, a combination of both electrical pulses 600a and electrical pulses 600b, or the like).

Continuing to block 804, routine 800 communicates the pulse width modulated control signal to an optical pump, wherein the optical pump, responsive to the pulse width modulated control signal, generates optical pump light and wherein a lasing medium, when exposed to the optical pump light, generates a laser beam. For example, controller 124 can send the pulse width modulated control signal to an optical pump 110 to cause the optical pump to generate optical pump light to excite lasing medium 114.

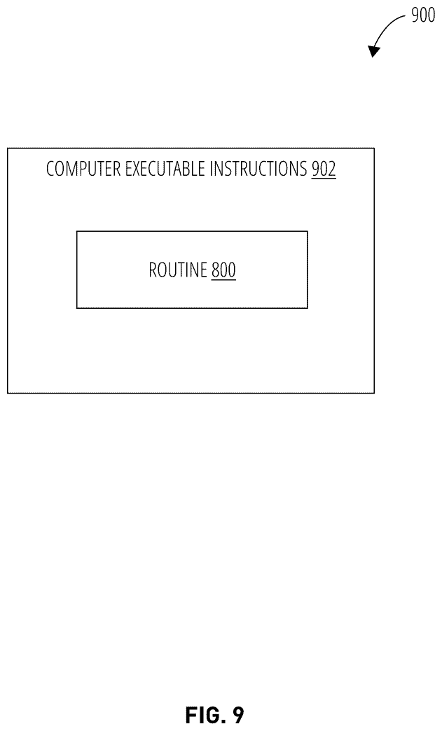

FIG. 9 illustrates computer-readable storage medium 900. Computer-readable storage medium 900 may comprise any non-transitory computer-readable storage medium or machine-readable storage medium, such as an optical, magnetic, or semiconductor storage medium. In various embodiments, computer-readable storage medium 900 may comprise an article of manufacture. In some embodiments, computer-readable storage medium 900 may store computer-executable instructions 902 with which circuitry (e.g., controller 124 or the like) can execute. For example, computer-executable instructions 902 can include instructions to implement operations described with respect to routine 800. Examples of computer-readable storage medium 900 or machine-readable storage medium may include any tangible media capable of storing electronic data, including volatile memory or non-volatile memory, removable or non-removable memory, erasable or non-erasable memory, writeable or re-writeable memory, and so forth. Examples of computer-executable instructions 902 may include any suitable type of code, such as source code, compiled code, interpreted code, executable code, static code, dynamic code, object-oriented code, visual code, and the like.

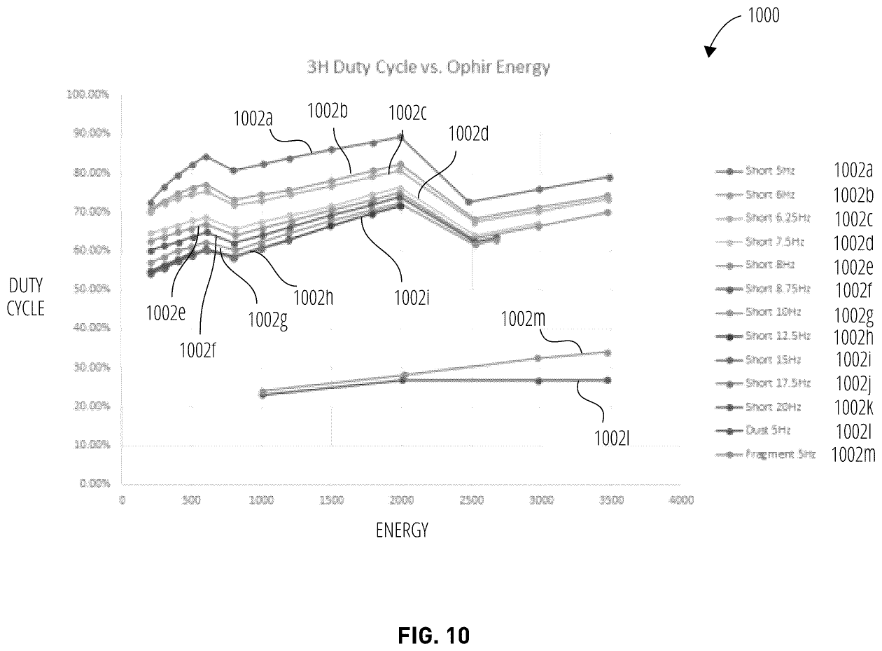

FIG. 10 illustrates graph 1000 depicting several PWM electrical pulses configured to excite an optical pump 110. The duty cycle of the PWM electrical pulses can be selected (and/or dynamically modified) such that the electrical pulse of laser beam 102 resulting from the excitation of optical pump 110 based on the depicted electrical pulses exhibits a reduced or dampened relaxation oscillations versus conventional electrical pulses used to excite optical pump 110.

As can be seen, graph 1000 depicts several plots 1002a, 1002b, 1002c, 1002d, 1002e, 1002f, 1002g, 1002h, 1002i, 1002j, 1002k, 1002l, and 1002m. Each of the plots 1002a to 1002m depicts the duty cycle (y-axis) vs. working beam packet energy (x-axis) created during open loop calibration of a cavity. During open loop calibration, the duty cycle is manually adjusted until the desired working beam packet energy is measured using an Ophir meter. The highest values of the duty cycle are used to generate working beam packets at a frequency of 5 Hz. The relationship between packet energy remains linear at other packet repetition frequencies.

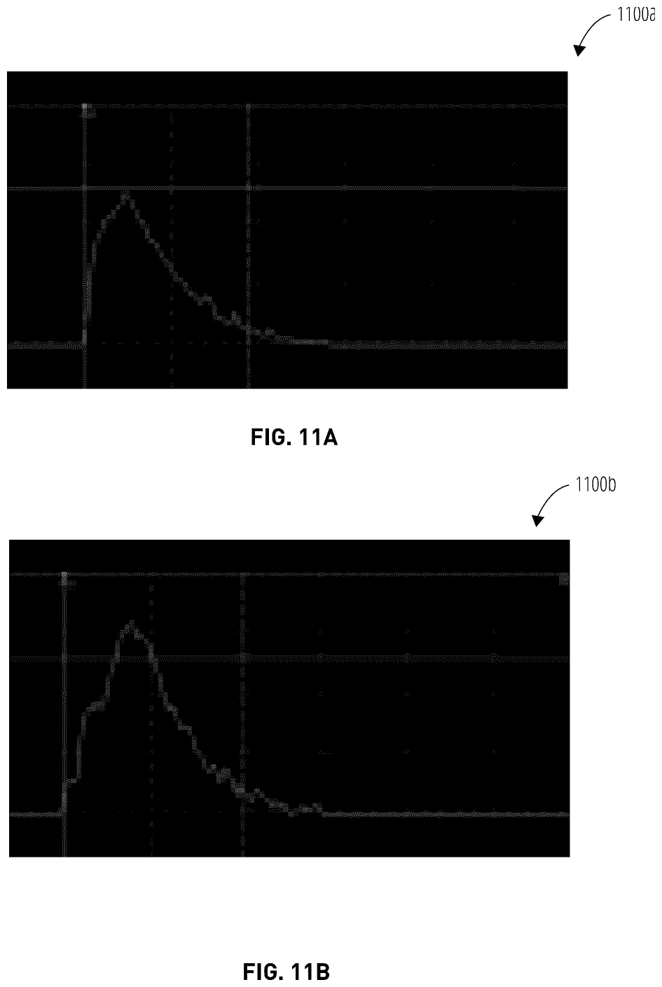

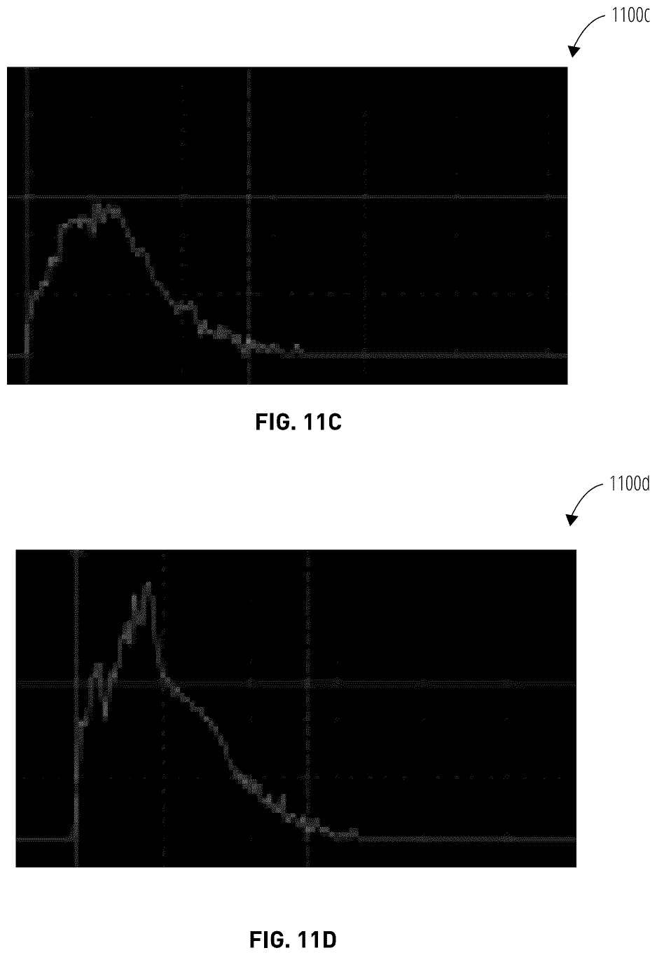

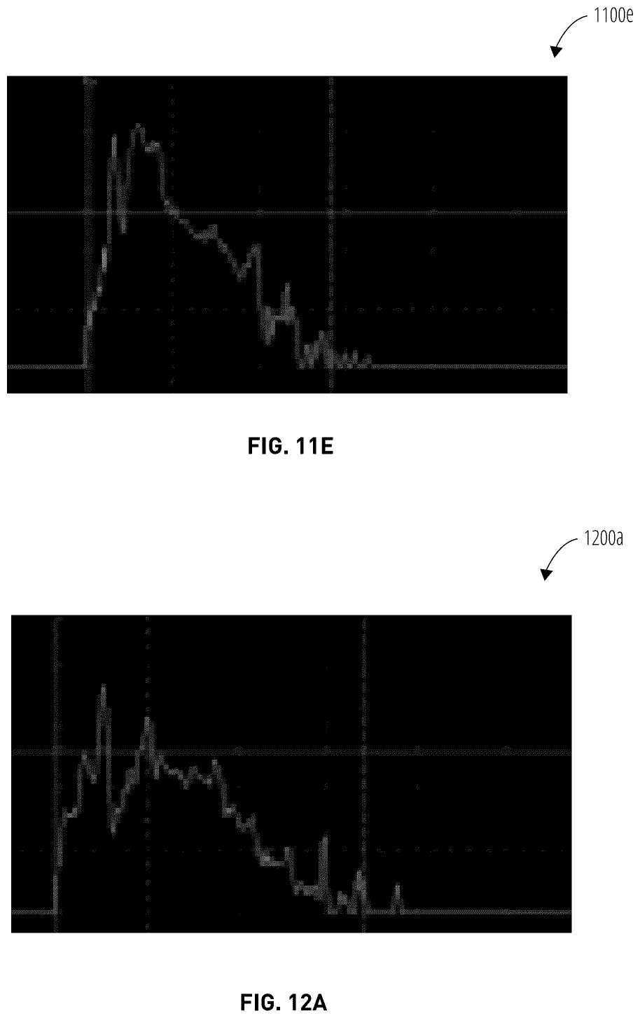

FIG. 11A to FIG. 11E illustrates oscilloscope captures of a photodiode signal for working beam packets for one of the plots in graph 1000 of FIG. 10 having small energies. FIG. 11A illustrates a pulse 1100a with packet energies of 200 millijoules (mJ). FIG. 11B illustrates a pulse 1100b with packet energies of 300 mJ. FIG. 11C illustrates a pulse 1100c with packet energies of 400 mJ. FIG. 11D illustrates a pulse 1100d with packet energies of 500 mJ. FIG. 11E illustrates a pulse 1100e with packet energies of 600 mJ. As can be seen from these pulses, as the PWM duty cycle and packet energy increase, a peak begins to appear on the leading edge of the pulse (e.g., working beam packet).

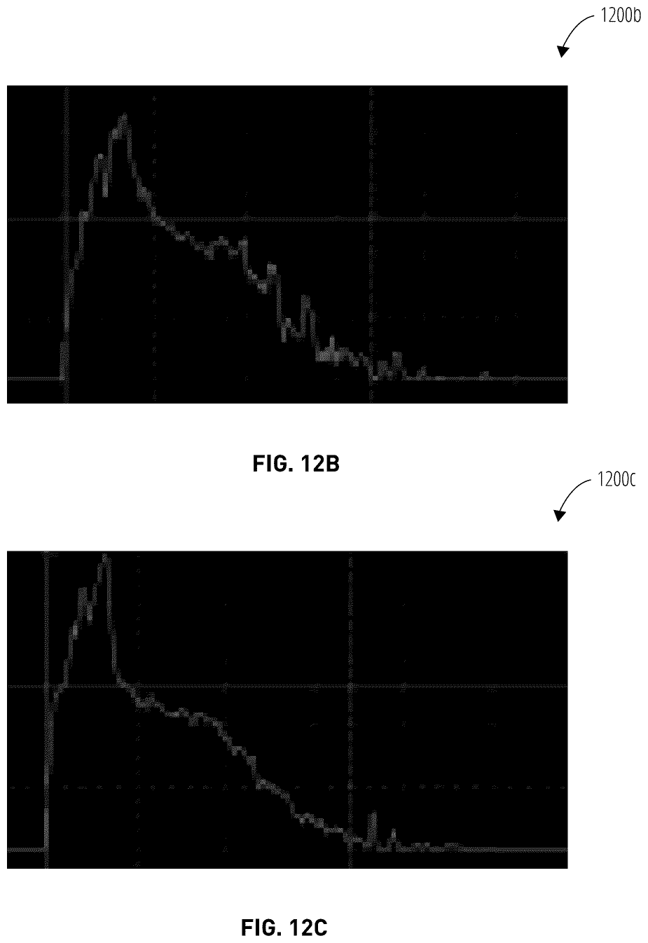

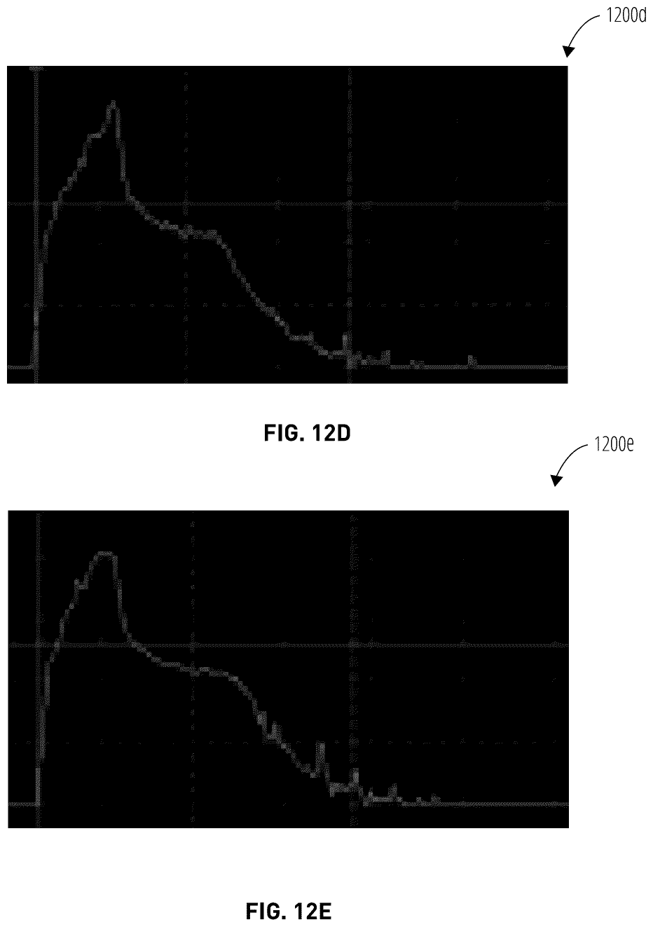

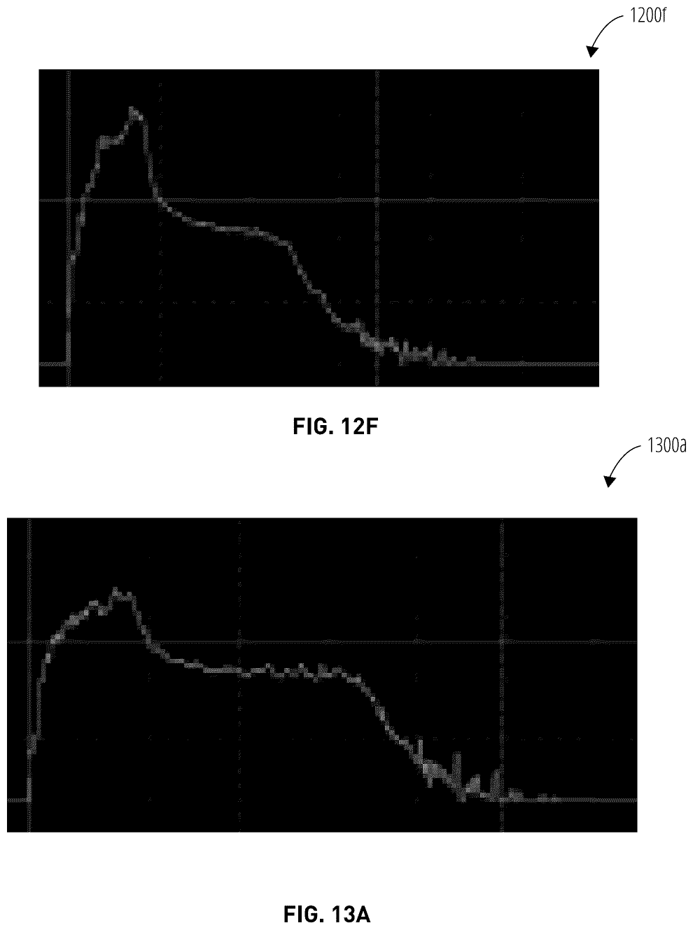

Similarly, FIG. 12A to FIG. 12F illustrates oscilloscope captures of a photodiode signal for working beam packets for one of the plots in graph 1000 of FIG. 10 having medium energies. FIG. 12A illustrates a pulse 1200a with packet energies of 800 mJ. FIG. 12B illustrates a pulse 1200b with packet energies of 1000 mJ. FIG. 12C illustrates a pulse 1200c with packet energies of 1200 mJ. FIG. 12D illustrates a pulse 1200d with packet energies of 1500 mJ. FIG. 12E illustrates a pulse 1200e with packet energies of 1800 mJ. FIG. 12F illustrates a pulse 1200f with packet energies of 2000 mJ. As can be seen from these pulses, as the PWM duty cycle and packet energy increase, a peak begins to appear on the leading edge of the pulse (e.g., working beam packet).

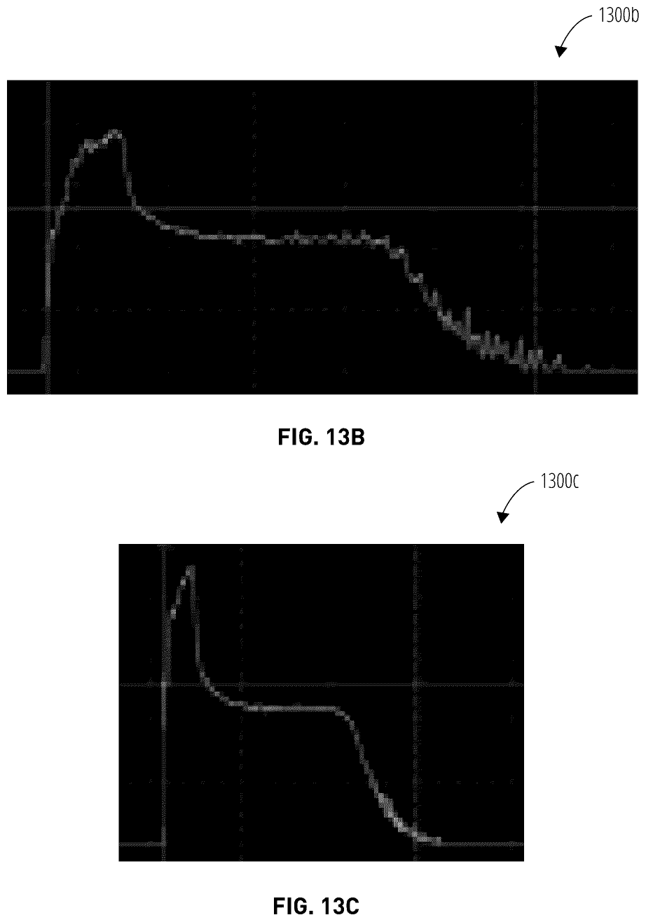

Finally, FIG. 13A to FIG. 13C illustrates oscilloscope captures of a photodiode signal for working beam packets for one of the plots in graph 1000 of FIG. 10 having large energies. FIG. 13A illustrates a pulse 1300a with packet energies of 2500 mJ. FIG. 13B illustrates a pulse 1300b with packet energies of 3000 mJ. FIG. 13C illustrates a pulse 1300c with packet energies of 3500 mJ. As can be seen from these pulses, as the PWM duty cycle and packet energy increase, a peak begins to appear on the leading edge of the pulse (e.g., working beam packet).

What is claimed is:

1. A laser system, comprising:
a controller configured to send a pulse width modulated control signal to one or more optical pumps to cause the optical pumps to generate optical pump light;
a lasing medium arranged to output a laser beam in response to the optical pump light; and
a pump chamber configured to direct the optical pump light to the lasing medium,
wherein the pulse width modulated control signal comprises a plurality of temporally spaced apart electrical pulse components configured to dampen a relaxation oscillation of the laser beam.

2. The laser system of claim 1, wherein the temporal delay between a first electrical pulse and a second electrical pulse of the plurality of electrical pulses is less than the temporal delay between the second electrical pulse and a third electrical pulse of the plurality of electrical pulses.

3. The laser system of claim 1, wherein a magnitude of a first electrical pulse is less than a magnitude of a subsequent electrical pulse of the plurality of electrical pulses.

4. The laser system of claim 1, wherein the temporal delay between a first electrical pulse and a second electrical pulse of the plurality of electrical pulses is less than the temporal delay between the second electrical pulse and a third electrical pulse of the plurality of electrical pulses, and wherein a magnitude of the first electrical pulse is less than a magnitude of the second electrical pulse of the plurality of electrical pulses.

5. The laser system of claim 1, wherein the frequency of the pulse width modulated control signal is between 4 and 25 kilohertz.

6. The laser system of claim 1, wherein the pulse width modulated control signal comprises a plurality of sets of electrical pulses, each set of electrical pulses of the plurality of sets of electrical pulses comprising a plurality of temporally spaced apart electrical pulses.

7. The laser system of claim 1, further comprising a saturable absorber disposed of in an optical path of the laser beam.

8. The laser system of claim 1, wherein the lasing medium includes one of Ho:YAG, Tm:YAG, Tm:Ho:YAG, Er:YAG, Er:YLF, Nd:YAG, Thulium fiber laser, and CTH:YAG.

9. A method for a laser system controller, comprising:
generating a pulse-width modulated control signal, the pulse-width modulated control signal comprising a plurality of electrical pulses separated by a temporal delay; and
communicating the pulse width modulated control signal to an optical pump, wherein the optical pump, responsive to the pulse width modulated control signal, generates optical pump light and wherein a lasing medium, when exposed to the optical pump light, generates a laser beam.

10. The method of claim 9, wherein the temporal delay between a first electrical pulse and a second electrical pulse of the plurality of electrical pulses is less than the temporal delay between the second electrical pulse and a third electrical pulse of the plurality of electrical pulses.

11. The method of claim 9, wherein a magnitude of a first electrical pulse is less than a magnitude of a subsequent electrical pulse of the plurality of electrical pulses.

12. The method of claim 9, wherein the temporal delay between a first electrical pulse and a second electrical pulse of the plurality of electrical pulses is less than the temporal delay between the second electrical pulse and a third electrical pulse of the plurality of electrical pulses, and wherein a magnitude of the first electrical pulse is less than a magnitude of the second electrical pulse of the plurality of electrical pulses.

13. The method of claim 9, wherein a frequency of the pulse width modulated control signal is between 4 and 25 kilohertz.

14. The method of claim 9, wherein the pulse width modulated control signal comprises a plurality of sets of electrical pulses, each set of electrical pulses of the plurality of sets of electrical pulses comprising a plurality of temporally spaced apart electrical pulses.

15. A non-transitory computer-readable storage medium, the computer-readable storage medium includes instructions that, when executed by a computer, cause the computer to:
generate a pulse-width modulated control signal, the pulse-width modulated control signal comprising a plurality of electrical pulses separated by a temporal delay; and
communicate the pulse width modulated control signal to an optical pump, wherein the optical pump, responsive to the pulse width modulated control signal, generates optical pump light and wherein a lasing medium, when exposed to the optical pump light, generates a laser beam.

16. The computer-readable storage medium of claim 15, wherein the temporal delay between a first electrical pulse and a second electrical pulse of the plurality of electrical pulses is less than the temporal delay between the second electrical pulse and a third electrical pulse of the plurality of electrical pulses.

17. The computer-readable storage medium of claim 15, wherein a magnitude of a first electrical pulse is less than that of a subsequent electrical pulse of the plurality of electrical pulses.

18. The computer-readable storage medium of claim 15, wherein the temporal delay between a first electrical pulse and a second electrical pulse of the plurality of electrical pulses is less than the temporal delay between the second electrical pulse and a third electrical pulse of the plurality of electrical pulses, and wherein a magnitude of the first electrical pulse is less than a magnitude of the second electrical pulse of the plurality of electrical pulses.

19. The computer-readable storage medium of claim 15, wherein the frequency of the pulse width modulated control signal is between 4 and 25 kilohertz.

20. The computer-readable storage medium of claim 15, wherein the pulse width modulated control signal comprises a plurality of sets of electrical pulses, each set of electrical pulses of the plurality of sets of electrical pulses comprising a plurality of temporally spaced apart electrical pulses.
